(19) United States
(12) Patent Application Publication
(10) Pub. No.: US 2014/0200675 A1
(43) Pub. Date: Jul. 17, 2014

(54) Lined Femoral Cup

(71) Applicant: Zafer Termanini, Cedar Grove, NJ (US)

(72) Inventor: Zafer Termanini, Cedar Grove, NJ (US)

(73) Assignee: Hip Innovation Technology, LLC, Plantation, FL (US)

(21) Appl. No.: 14/237,588

(22) PCT Filed: Jul. 18, 2012
(86) PCT No.: PCT/US2012/047164

§ 371 Date: Feb. 7, 2014

Related U.S. Application Data
(60) Provisional application No. 61/529,955, filed on Sep. 1, 2011.

(51) Int. Cl. A61F 2/36

(52) U.S. Cl. CPC A61F 2/3609
USPC 623/23.13

(57) ABSTRACT

The lined femoral cup of the invention is used in a new reverse hip prosthesis wherein the cup is affixed to the femur and the ball is affixed to the acetabulum. This permits installation of the liner in the factory instead of the operating room. Prior art hip prostheses require installation of the liner in the operating room because the cup must be affixed to the acetabulum, usually by screws, before the liner can be installed. Thus, in a preferred embodiment of the invention the liner is installed under high pressure in the factory. In a second embodiment, a threaded cap is used which permits replacement of the liner without removing the cup from the patient.

### BACKGROUND OF THE INVENTION

1. Field of the Invention

The present invention has to do with a lined femoral cup assembly used in a reverse hip prosthesis. More specifically, the invention relates to a new plastic liner and, in another embodiment, a removable plastic or ceramic liner for a new femoral cup used in the new hip replacement prosthesis described in U.S. patent application Ser. No. 13/024,381 filed Feb. 10, 2011 entitled “Interlocking Reverse Hip and Revision Prosthesis” and its parent application Ser. No. 12/799,609 filed on Apr. 28, 2010 and Provisional Application Ser. No. 61/339,680 filed on Mar. 8, 2010, the disclosures of all of which are incorporated herein by reference,

2. The Related Art

The prior art discloses cups and liners used in hip replacement prostheses. But the lined cups of the prior art are acetabular cups and the liners must be affixed in the cups during surgery because the acetabular cup needs to be affixed to the acetabulum, such as by screws, before the liner can be installed. For example, U.S. Patent Application Publication No. 2011/0015753 A1 discloses a polyethylene liner which is aligned with and affixed into an acetabular cup by the surgeon during hip replacement surgery.

### SUMMARY OF THE INVENTION

In a first embodiment, the present invention is an improvement over the art not only because the prosthesis of the invention is an improvement but also because the liner of the invention is affixed in the femoral cup as a part of the manufacturing process thereby eliminating a step in the operating room and making a substantially more secure, machine pressed connection between the liner and the cup.

In a second embodiment of the present invention, the femoral cup has a removable threaded cap element which permits removal and replacement of the liner. The liner employed in this embodiment can be comprised of the same type of plastic as is used in the first embodiment or a hard material such as a ceramic.

The plastic and ceramic materials that are employed in the present invention are well known to those having skill in the art.

### BRIEF DESCRIPTION OF THE DRAWINGS

The mechanical elements of the invention are illustrated in the drawings and are summarized as follows:

FIG. 1 is a section view of the first embodiment of the lined femoral cup assembly of the invention.

FIG. 2 is a section view of the femoral cup of FIG. 1 without the liner.

FIG. 3A is a top elevation view of the plastic liner.

FIG. 3B is a section view taken through section line 3B-3B of FIG. 3A.

FIG. 3C is a side elevation view of the liner of FIG. 3A.

FIG. 3D is an expanded section view of the portion of the liner encircled by circle 3D in FIG. 3B.

FIG. 3E is a bottom elevation view of the liner of FIGS. 3A and 3C.

FIG. 4A is a side elevation view of a femoral cup of the invention.

FIG. 4B is a section view taken through section line 4B-4B of FIG. 4A and illustrating in section a variation of the first embodiment of the invention.

FIG. 4C is a top elevation view of FIG. 4A.

FIG. 5A is a side elevation view of the liner of the FIG. 4B embodiment of the invention.

FIG. 5B is a top elevation view of the liner of FIG. 5A.

FIG. 5C is a bottom elevation view of the liner of FIGS. 5A and 5B.

FIG. 6 is a partial section view of the prosthesis of the invention employing the femoral cup and liner of the FIG. 4B embodiment.

FIG. 7 is a section view of the second embodiment of the invention.

FIG. 8 is an exploded view of FIG. 7.

### DETAILED DESCRIPTION OF THE INVENTION

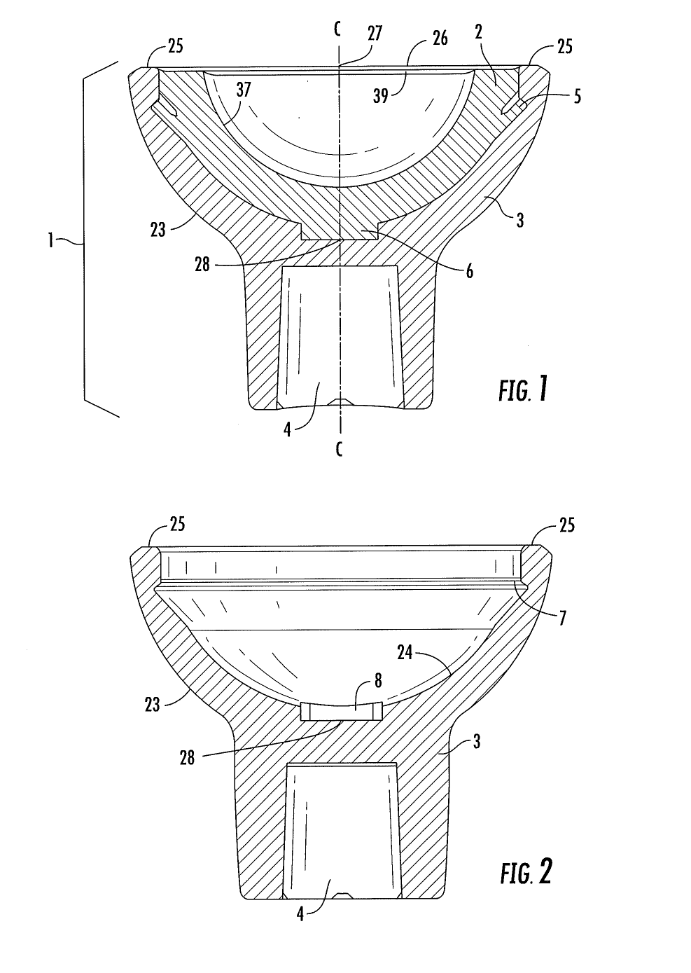

Referring to the drawings, FIG. 1 illustrates in section a femoral cup assembly 1 comprised of a liner 2 which was factory installed under high pressure into femoral cup 3 using an Arbor press. A preferred material for the liner is polyethylene but other materials can be used as will be apparent to those having skill in the art. Recess 4 is sized to receive the stem of a femoral implant. The recess 4 has a Morse taper, as does the stem. The femoral implant is for implantation into the medullary canal of a proximal end of a femur.

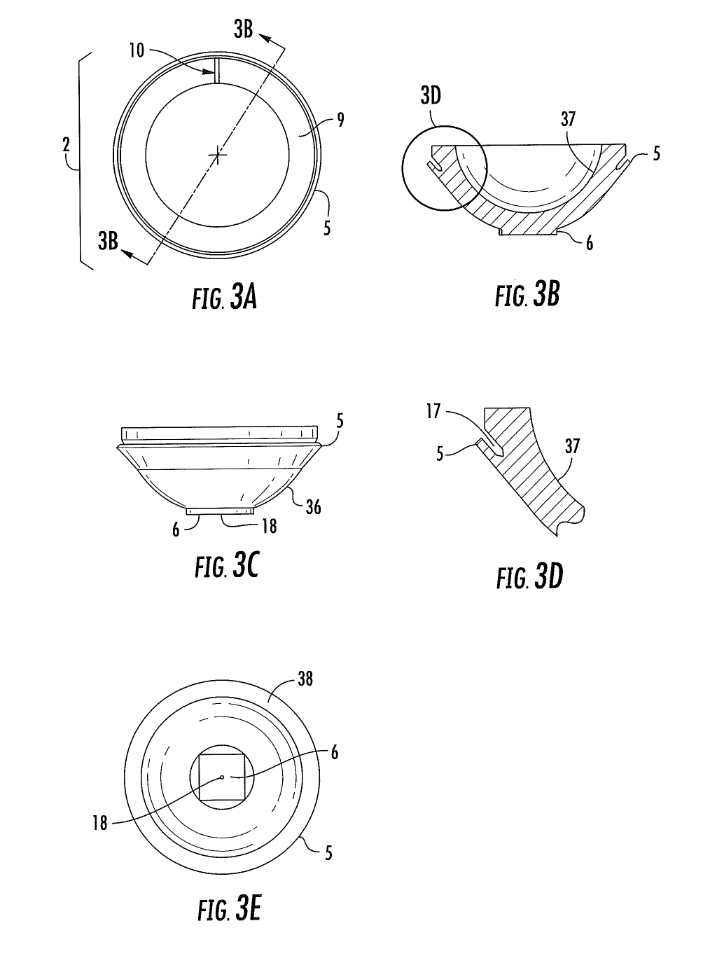

Referring to FIGS. 1 and 2, FIG. 2 being a section view of femoral cup 3 without liner 2, annular protrusion 5, a continuous circumferential skirt, is a locking element which mates with annular groove 7. Locking protrusion 6, which is centered about the apex of the outer convex liner surface, mates with locking recess 8. Locking recess 8 is centered about the perigee or bottom point 28 of the inner concave surface of femoral cup 3. The locking protrusion 6 and locking recess 8 are illustrated in the shape of a square with rounded edges (see FIG. 3E) but other shapes that prevent rotation of the liner relative to the cup can be used. The annular protrusion 5 and annular groove 7 prevent lateral movement of the liner relative to the cup. The locking protrusion 6 and the locking recess 8 prevent both rotational and lateral movement of the liner relative to the cup.

The femoral cup 3 has an outer convex surface 23 and an inner concave surface 24. Annular edge 25 extends between the outer convex surface 23 and the inner concave surface 24. The annular edge 25 defines a first plane 26 and the first plane has a center point 27. A center line C-C, perpendicular to the first plane, extends through center point 27 and bottom point 28. The bottom point 28 is at the greatest distance along center line C-C from the center point 27 and within the concave surface 24.

The liner 2 has an outer convex liner surface 36 and an inner concave liner surface 37. Annular liner edge 38 extends between the outer convex liner surface 36 and the inner concave liner surface 37. The annular liner edge 38 defines a second plane 39. The second plane 39 can be the same as (coplanar with) the first plane 26 or it can be in close proximity to the first plane 26 and in parallel with or approximately in parallel with the first plane 26. The outer convex liner surface 36 is shaped and sized the same as or about the same as the inner concave surface 24 so that the outer convex liner surface 36 fits in mating relation with the inner concave surface 24 in a secure, machine pressed connection within cup 3.

The outer convex liner surface 36 has an apex 18 (see FIGS. 3C and 3E.) and centered about the apex 18 is the locking protrusion 6 which is sized to mate with locking recess 8. When liner 2 is pressed into cup 3, annular gap 17 allows the annular protrusion 5 to flex slightly inwardly as the annular protrusion 5 passes annular cup edge 25. With continued downward pressure on liner 2, the locking protrusion 6 is seated in locking recess 8 and the annular protrusion 5 snaps outwardly causing annular protrusion 5 to tightly and lockingly engage annular groove 7.

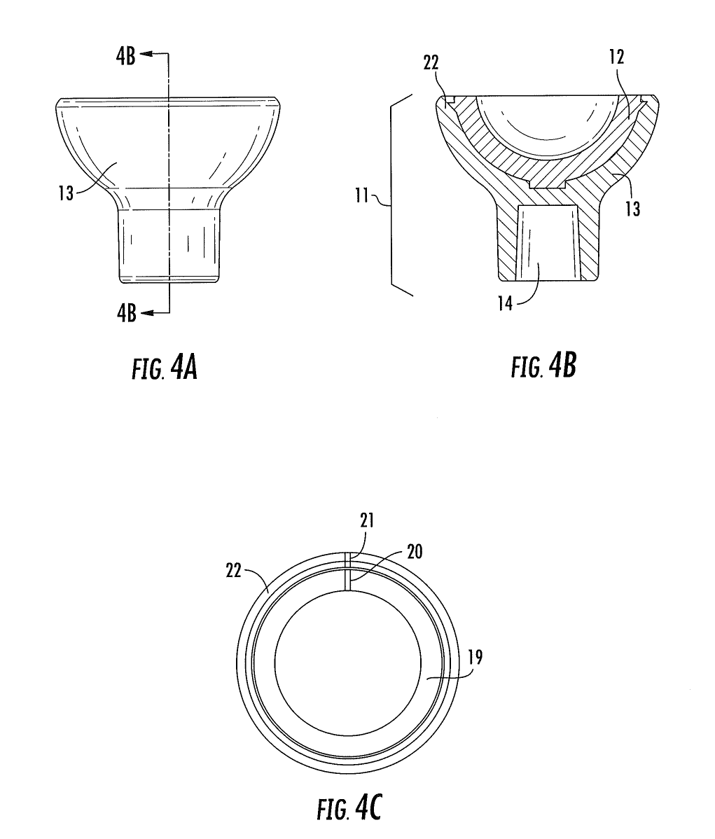

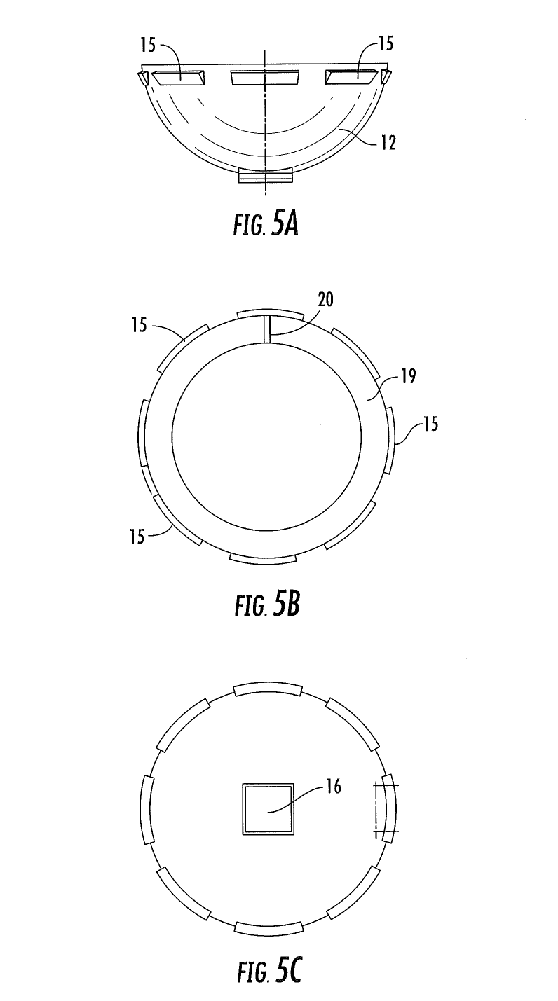

FIGS. 3A-3 E illustrate liner 2 in more detail. The top elevation view of FIG. 3A illustrates annular liner edge 9 and liner assembly mark 10. The liner assembly mark 10 is lined up with a mark on the femoral cup so that the liner 2 is properly lined up with the femoral cup before it is pressed into the cup. (This is illustrated in FIG. 4C in respect of the variation of the first embodiment.) Section view 3B and expanded partial section view 3D illustrate annular protrusion 5 in more detail. FIG. 3C makes it clear that annular protrusion 5 is in the shape of a continuous circumferential skirt. Because the skirt is continuous, considerable pressure is needed to snap fit the liner 2 into femoral cup 3. This is done with a press during manufacture. As explained above, the compression causes annular protrusion 5 to be deflected into annular gap 17 so that liner 2 is snap-fitted under pressure in femoral cup 3 when the liner 2 is sufficiently pressed into cup 3 to allow annular protrusion 5 to snap back to its original position. The compression results in a very stable and tight pressurized fit that is not achievable when a surgeon installs a liner in the operating room.

FIG. 3E provides a more detailed illustration of locking protrusion 6.

FIGS. 4-6 illustrate a variation of the first embodiment of the invention which employs locking tabs 15 instead of the annular protrusion 5 of the first embodiment. As can be seen from the drawings, locking tabs 15 extend outwardly form the outer convex liner surface. The tabs 15 are spaced from one another and are arranged annularly around said outer convex liner surface. The tabs 15 lie in proximity to and below annular liner edge 19, each tab being located the same distance or about the same distance below liner edge 19.

Side elevation view FIG. 4A illustrates femoral cup 13. FIG. 4B is a section view of FIG. 4A taken along section line 4B-4B. And femoral cup assembly 11 is depicted in FIG. 4B with liner 12, recess 14 and annular cup edge 22.

Top elevation view FIG. 4C illustrates liner assembly mark 20 on annular liner edge 19. The liner assembly mark 20 is lined up with cup assembly mark 21 on annular cup edge 22.

FIG. 5A is a side elevation view of liner 12 with locking tabs 15. The top elevation view, FIG. 5B, depicts the locking tabs 15 spaced around the perimeter of liner 12 and bottom elevation view 5C illustrates locking protrusion 16.

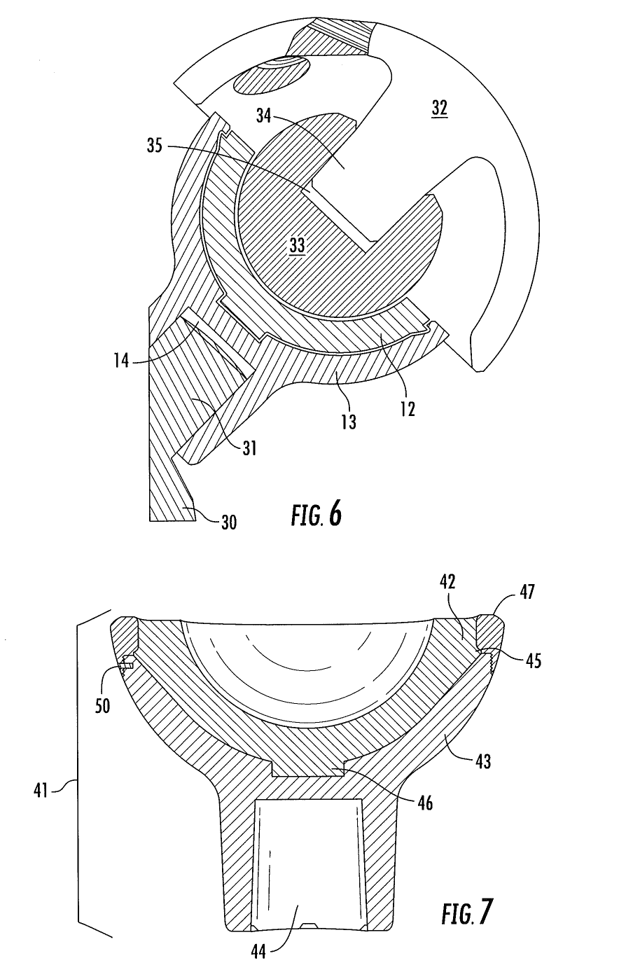

FIG. 6 is a section view of the variation of the first embodiment of the liner assembly illustrating the liner assembly 11 installed in the reverse hip prosthesis. In addition to the elements discussed above, a portion of a femoral implant 30 is shown with stem 31 affixed in recess 14. Element 32 is an acetabular cup and acetabular ball 33 is affixed to the stem 34 thereof. Stem 34 has a Morse taper sized to mate with ball recess 35. The inner concave surface of liner 12 articulates on the surface of acetabular ball 33.

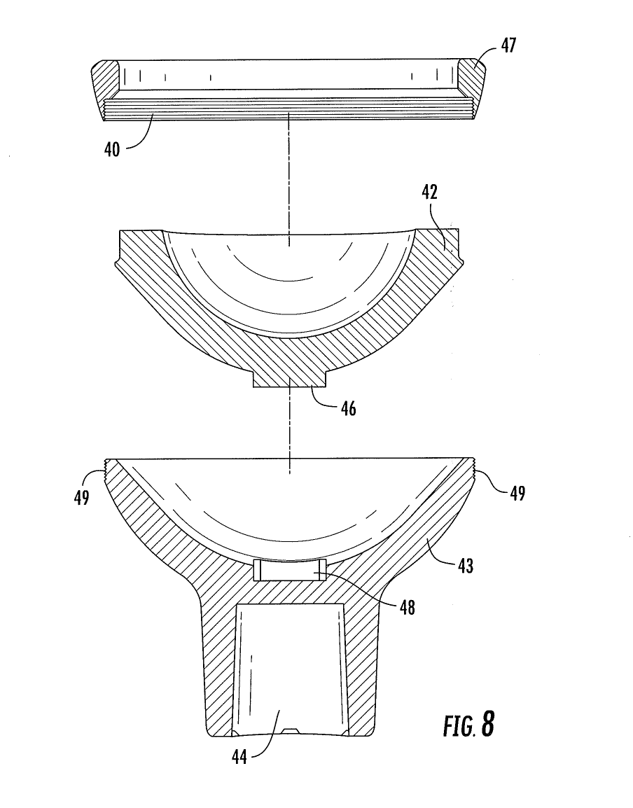

FIGS. 7 and 8 illustrate in section a second embodiment of the invention. Femoral cup assembly 41 is comprised of liner 42 which is removably installed in femoral cup 43. Annular locking cap 47 comprises threads 48 which mate with threads 49 on cup 43. The assembly 41 is put together by inserting liner 42 into cup 43 and turning the liner to mate locking protrusion 46 with locking recess 48. Then annular locking cap 47 is screwed tightly onto cup 43 to hold liner 42 in place. The liner 42 can be removed and replaced by unscrewing locking cap 47. One or more than one optional screw or pin 50 can be used to prevent locking cap 47 from coming loose.

What is claimed is:

1. A femoral cup assembly comprising
a femoral cup having an outer convex surface, an inner concave surface and an annular cup edge extending between the outer convex surface and the inner concave surface, the annular cup edge defining a first plane, the first plane having a center point, the outer convex surface having a stem or recess sized to receive a femoral implant,
the inner concave surface having a bottom point located at the greatest distance along a center line from the center point, the center line being perpendicular to the first plane, a annular groove located in proximity to the annular cup edge and a locking recess centered about the bottom point,
a liner affixed in the inner concave surface of the femoral cup, the liner having an outer convex liner surface, an inner concave liner surface and an annular liner edge extending between the outer convex liner surface and the inner concave liner surface, the annular liner edge defining a second plane, the second plane being the same as the first plane or lying in proximity to and in parallel with or approximately in parallel with the first plane,
the outer convex liner surface having a shape and size the same as or about the same as the shape and size of the inner concave surface of the femoral cup,
the outer convex liner surface further having an apex and centered about the apex a locking protrusion sized to mate in a first locking relation with the locking recess, and an annular protrusion located in proximity to the annular liner edge and sized and shaped to mate in a second locking relation with the annular groove.

2. The femoral cup assembly of claim 1 wherein the annular protrusion is a continuous circumferential skirt.

3. The femoral cup assembly of claim 1 wherein the liner is under pressure in the femoral cup.

4. A femoral cup assembly comprising
a femoral cup having an outer convex surface, an inner concave surface and an annular cup edge extending between the outer convex surface and the inner concave surface, the annular cup edge defining a first plane, the first plane having a center point, the outer convex surface having a stem or recess sized to receive a femoral implant,
the inner concave surface having a bottom point located at the greatest distance along a center line from the center point, the center line being perpendicular to the first plane, an annular groove located in proximity to the annular cup edge and a locking recess centered about the bottom point,
a liner affixed in the inner concave surface of the femoral cup, the liner having an outer convex liner surface, an inner concave liner surface and an annular liner edge extending between the outer convex liner surface and the inner concave liner surface, the annular liner edge defining a second plane, the second plane being the same as the first plane or lying in proximity to and in parallel with or approximately in parallel with the first plane,
the outer convex liner surface having a shape and size the same as or about the same as the shape and size of the inner concave surface of the femoral cup,
the outer convex liner surface further having an apex and centered about the apex a locking protrusion sized to mate in a first locking relation with the locking recess, and spaced locking tabs extending outwardly from the outer convex liner surface and annularly around the outer convex liner surface, the locking tabs being located in proximity to the annular liner edge and sized and shaped to mate in a second locking relation with the annular groove.

5. A femoral cup assembly comprising
a femoral cup having an outer convex surface with a threaded upper rim, an inner concave surface and a cap having a threaded portion matingly threaded onto the threaded upper rim, the cap having an annular cup edge which extends between the outer convex surface and the inner concave surface when the cap is threaded onto the threaded upper rim, the annular cup edge defining a first plane, the first plane having a center point, the outer convex surface having a stem or recess sized to receive a femoral implant,
the inner concave surface having a bottom point located at the greatest distance along a center line from the center point, the center line being perpendicular to the first plane, an annular groove located in proximity to the annular cup edge and a locking recess centered about the bottom point,
a liner affixed in the inner concave surface of the femoral cup, the liner having an outer convex liner surface, an inner concave liner surface and an annular liner edge extending between the outer convex liner surface and the inner concave liner surface, the annular liner edge defining a second plane, the second plane being coplanar with the first plane or lying in proximity to and in parallel with or approximately in parallel with the first plane,
the outer convex liner surface having a shape and a size the same as or about the same as the shape and size of the inner concave surface of the femoral cup,
the outer convex liner surface further having an apex and centered about the apex a locking protrusion sized to mate in a first locking relation with the locking recess, and an annular protrusion located in proximity to the annular liner edge and sized and shaped to mate in a second locking relation with the annular groove.

6. The femoral cup assembly of claim 5 wherein the annular protrusion is a continuous circumferential skirt.

7. The femoral cup assembly of claim 5 wherein the annular protrusion is comprised of spaced locking tabs extending outwardly from the outer convex liner surface and annularly around the outer convex surface.

8. A liner for a femoral cup, the liner having an outer convex liner surface, an inner concave liner surface and an annular liner edge extending between the outer convex liner surface and the inner concave liner surface,
the outer convex liner surface being shaped and sized to fit matingly in an inner concave surface of a femoral cup,
the outer convex liner surface further having an apex and centered about the apex a locking protrusion sized to mate in a first locking relation with a locking recess in the femoral cup, and a continuous circumferential skirt protrusion located in proximity to the annular cup edge and sized and shaped to mate in a second locking relation with an annular groove in the femoral cup.

9. A liner for a femoral cup, the liner having an outer convex liner surface, an inner concave liner surface and an annular liner edge extending between the outer convex liner surface and the inner concave liner surface,
the outer convex liner surface being shaped and sized to fit matingly in an inner concave surface of a femoral cup,
the outer convex liner surface further having an apex and centered about the apex a locking protrusion sized to mate in a first locking relation with a locking recess in the femoral cup, and spaced locking tabs extending outwardly from the outer convex liner surface and annularly around the outer convex liner surface, the locking tabs being located in proximity to the annular liner edge and sized and shaped to mate in a second locking relation with an annular groove in the femoral cup.
